(19) United States
(12) Patent Application Publication
(10) Pub. No.: US 2011/0021547 A1
(43) Pub. Date: Jan. 27, 2011

(54) Substantially Pure and a Stable Crystalline Form of Bosentan

(75) Inventors: Girish Dixit, Ghaziabad (IN); Nandkumar Gaikwad, Maharashtra (IN); Hima Prasad Naidu, Andhra Pradesh (IN); Nitin Sharadchandra Pradhan, Maharashtra (IN); Jon Valgeirsson, Hafnarfjordur (IS)

Correspondence Address: CANTOR COLBURN LLP, 20 Church Street, 22nd Floor, Hartford, CT, 06103, US

(73) Assignee: ACTAVIS GROUP PTC EHF, Hafnarfjordur (IS)

(21) Appl. No.: 12/864,571

(22) PCT Filed: Jan. 22, 2009
(86) PCT No.: PCT/IB09/00108

§ 371 Date: Oct. 4, 2010

(30) Foreign Application Priority Data
Jan. 24, 2008 (IN) 197/CHE/2008
Mar. 13, 2008 (IN) 628/CHE/2008
Mar. 18, 2008 (IN) 675/CHE/2008

(51) Int. Cl. A61K 31/513; C07D 403/04

(52) U.S. Cl. 514/269; 544/296

(57) ABSTRACT

Described is a highly stable crystalline form of bosentan having a water content in the range of about 3-4% by weight, based on the total weight of the bosentan, (bosentan crystalline form A5), a process for preparation thereof, and pharmaceutical compositions comprising the bosentan crystalline form A₅. Provided also herein is a bosentan impurity, p-tert-butyl-N[6-hydroxy-5-(2-methoxyphenoxy)-2-(2-pyrimidinyl)-4-pyrimidinyl]benzenesulfonamide (deshydroxyethyl bosentan impurity), and process for preparing and isolating thereof. Further provided are highly pure bosentan or a pharmaceutically acceptable salt thereof substantially free of deshydroxyethyl bosentan and bosentan dimer impurities, process for the preparation thereof, and pharmaceutical compositions comprising solid particles of highly pure bosentan or a pharmaceutically acceptable salt thereof, wherein 90 volume-percent of the particles (D₉₀) have a size of less than about 300 microns.

### CROSS REFERENCE TO RELATED APPLICATION

This application claims the benefit of priority to Indian provisional application Nos. 197/CHE/2008, filed on Jan. 24, 2008; 628/CHE/2008, filed on Mar. 13, 2008; and 675/CHE/2008, filed on Mar. 18, 2008; which are incorporated herein by reference.

### FIELD OF THE INVENTION

The present invention relates to a novel and stable crystalline form of bosentan, a process for the preparation thereof, and pharmaceutical compositions comprising the bosentan crystalline form. The present invention also relates to the bosentan impurity, p-tert-butyl-N-[6-hydroxy-5-(2-methoxyphenoxy)-2-(2-pyrimidinyl)-4-pyrimidinyl]benzenesulfonamide (hereinafter referred to as the ‘deshydroxyethyl bosentan impurity’), and process for preparing and isolating thereof. The present invention further provides highly pure bosentan or a pharmaceutically acceptable salt thereof substantially free of deshydroxyethyl bosentan and bosentan dimer impurities, and process for the preparation thereof. The present invention further relates to pharmaceutical compositions comprising solid particles of highly pure bosentan or a pharmaceutically acceptable salt thereof, wherein 90 volume-percent of the particles (D₉₀) have a size of less than about 300 microns.

### BACKGROUND OF THE INVENTION

U.S. Pat. No. 5,292,740 discloses a variety of sulfonamide derivatives, processes for the preparation, pharmaceutical compositions and method of use thereof. These compounds are useful in treatment of a variety of illness including cardiovascular disorders such as hypertension, ischemia, vasospasms and angina pectoris. Among them, Bosentan, p-tert-butyl-N-[6-(2-hydroxyethoxy)-5-(2-methoxyphenoxy)-2-(2-pyrimidinyl)-4-pyrimidinyl]benzenesulfonamide monohydrate, has a wide variety of biological activities including inhibiting the renin angiotensin system and acting as an endothelin antagonist. Bosentan blocks the binding of endothelin to its receptors, thereby negating endothelin's deleterious effects. Bosentan has the molecular formula of C₂₇H₂₉N₅O₆S.H₂O, molecular weight of 569.63 and a structural formula of:

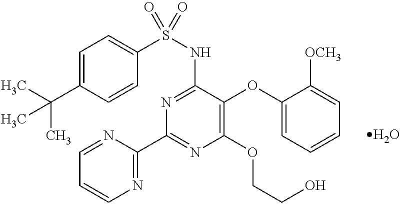

Various processes for the preparation of Bosentan and related compounds were disclosed in U.S. Pat. No. 5,292,740 and U.S. Pat. No. 6,136,971.

According to the U.S. Pat. No. 5,292,740 (hereinafter referred to as the '740 patent), bosentan is prepared by the reaction of 5-(2-methoxyphenoxy)-2-(2-pyrimidin-2-yl)-4,6(1H,5H)-pyrimidinedione with phosphorous oxychloride in acetonitrile to give 4,6-dichloro-5-(2-methoxyphenoxy)-2,2′-bipyrimidine, which by condensation with 4-tert-butylbenzenesulfonamide potassium in dimethylsulfoxide followed by treatment with hydrochloric acid to afford p-tert-butyl-N-[6-chloro-5-(2-methoxyphenoxy)-2-(2-pyrimidinyl)-4-pyrimidinyl]benzenesulfonamide, which is then reacted with a sodium ethylene glycol, prepared by the reaction of ethylene glycol and sodium metal, in ethylene glycol solvent to produce bosentan as sodium salt (m.p. 195-198° C.). The '740 patent involve the use of sodium metal for the preparation of sodium ethylene glycolate. Sodium metal is explosive and hazardous reagent and vigorously reacts with water. The use of sodium metal is not advisable for scale up operations. Moreover, the bosentan obtained by the process described in the '740 patent by using sodium metal is not satisfactory from purity point of view. Unacceptable amounts of impurities are generally formed along with bosentan.

According to the U.S. Pat. No. 6,136,971 (hereinafter referred to as the '971 patent), bosentan is prepared by the reaction of 5-(2-methoxyphenoxy)-2-(2-pyrimidin-2-yl)-4,6(1H,5H)-pyrimidinedione with phosphorous oxychloride in toluene to give 4,6-dichloro-5-(2-methoxyphenoxy)-2,2′-bipyrimidine, which by condensation with 4-tert-butylbenzenesulfonamide in the presence of anhydrous potassium carbonate and a phase transfer catalyst (e.g., benzyltriethylammonium chloride) in toluene to get p-tert-butyl-N-[6-chloro-5-(2-methoxyphenoxy)-2-(2-pyrimidinyl)-4-pyrimidinyl]benzenesulfonamide potassium salt, which is then reacted with ethylene glycol mono-tert-butyl ether in toluene in the presence of granular sodium hydroxide to give p-tert-butyl-N-[6-(2-tert-butyl-ethoxy)-5-(2-methoxyphenoxy)[2,2′-bipyrimidin]-4-yl]benzene-sulfonamide (Bosentan tert-butyl ether). The Bosentan tert-butyl ether obtained is then reacted with formic acid followed by treatment with absolute ethanol to afford bosentan formate monoethanolate, which by reaction with sodium hydroxide in absolute ethanol and water followed by acidification with hydrochloric acid and then the resulting precipitate is suction-filtered, washed with ethanol-water mixture (1:1) to give Bosentan crude. The crude bosentan obtained is then purified with mixture of ethanol and water and the resulting precipitate is suction-filtered to give bosentan.

The '971 patent makes no reference to the existence of specific polymorphic forms of bosentan. The synthetic route of bosentan described in the '971 patent involves lengthy process, and the yields are very low.

PCT publication No. WO 2008/135795 (herein after referred to as the '795 application) discloses four crystalline forms (forms 1, 2, 3 & 4) and an amorphous form of bosentan, characterizes them by powder X-ray diffraction (P-XRD), Differential Scanning Calorimetry (DSC), and Thermogravimetric Analysis (TGA). According the '795 application, the crystalline form 1 is characterized by an X-ray powder diffraction pattern having peaks expressed as 2-theta at about 3.9, 7.8, 8.8, 13.2, 16.1, 17.6, 18.7, 23.0 and 24.0±0.2 degrees, and a DSC thermogram comprising an endotherm at about 148° C.; the crystalline form 2 is characterized by an X-ray powder diffraction pattern having peaks expressed as 2-theta at about 7.6, 13.6, 16.6, 16.9, 17.3, 18.6, 20.0, 20.3 and 23.0±0.2 degrees, and a DSC thermogram comprising an endotherm at about 144° C.; the crystalline form 3 is characterized by an X-ray powder diffraction pattern having peaks expressed as 2-theta at about 5.2, 7.5, 8.2, 9.3, 10.0, 18.1, 20.5, 21.5 and 25.0±0.2 degrees, and a DSC thermogram comprising an endotherm at about 174° C.; and the crystalline form 4 is characterized by an X-ray powder diffraction pattern having peaks expressed as 2-theta at about 5.7, 6.4, 9.5, 15.6, 16.6, 21.2, 21.5, 27.4 and 31.8±0.2 degrees, and a DSC thermogram comprising an endotherm at about 210° C.

Polymorphism is defined as “the ability of a substance to exist as two or more crystalline phases that have different arrangement and/or conformations of the molecule in the crystal lattice. Thus, in the strict sense, polymorphs are different crystalline forms of the same pure substance in which the molecules have different arrangements and/or configurations of the molecules”. Different polymorphs may differ in their physical properties such as melting point, solubility, X-ray diffraction patterns, and the like. Although those differences disappear once the compound is dissolved, they can appreciably influence the pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and infrared spectrometry (IR).

Solvent medium and mode of isolation play very important role in obtaining a polymorphic form over the other.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

We have now surprisingly and unexpectedly found a novel polymorphic form of bosentan, different from the material obtained according to the teachings of the '971 patent and the polymorphic forms disclosed in the '795 application, and having adequate stability and good dissolution properties.

In our hands, the methods of the '971 patent yield a crystalline form, which we denote as Form I, characterized by an X-ray powder diffraction pattern having peaks expressed as 2-theta angle positions at about 6.34, 10.77, 12.69, 15.85, 19.05, 19.84 and 21.29±0.2 degrees, different from the crystal form of the present invention.

Organic Process Research & Development 2002, 6, 120-124 discloses that the bosentan obtained as per the synthetic route described in the '740 patent is generally not satisfactory purity, unacceptable amounts of impurities are generally formed along with Bosentan. Hence it required three further crystallizations to provide specification grade bosentan suitable for formulation.

It is known in the art that any synthetic compound can contain extraneous compounds or impurities that can come from various sources. They can be unreacted starting materials, by-products of the reaction, products of side reactions, or degradation products. Generally, impurities in an active pharmaceutical ingredient (API) may arise from degradation of the API itself, or during the preparation of the API. Impurities in bosentan or any active pharmaceutical ingredient (API) are undesirable and might be harmful.

Regulatory authorities worldwide require that drug manufactures isolate, identify and characterize the impurities in their products. Furthermore, it is required to control the levels of these impurities in the final drug compound obtained by the manufacturing process and to ensure that the impurity is present in the lowest possible levels, even if structure determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the API, it must be analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. As discussed above, in the United States, the Food and Drug Administration guidelines recommend that the amounts of some impurities limited to less than 0.1 percent.

Generally, impurities are identified spectroscopically and by other physical methods and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRt”) to identify impurities. The RRt of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product. The present invention relates to an impurity of bosentan, p-tert-butyl-N-[6-hydroxy-5-(2-methoxyphenoxy)-2-(2-pyrimidinyl)-4-pyrimidinyl]benzenesulfonamide, designated, ‘deshydroxyethyl bosentan impurity’, whose presence was observed in bosentan and it has not been reported in the literature. The deshydroxyethyl bosentan impurity has the following structural formula I:

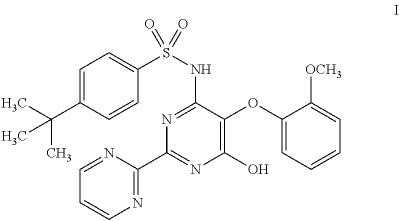

and it is identified, isolated and synthesized. The deshydroxyethyl bosentan impurity is detected and resolved from bosentan by HPLC with an RRt of 0.95. The structure of the deshydroxyethyl bosentan impurity was deduced with the aid of ¹H, ¹³C NMR, IR spectroscopy and FAB mass spectrometry. The parent ion at 507.5 is consistent with assigned structure.

The '971 patent further discloses a dimer impurity of bosentan. The '971 patent teaches that, the process for the preparation of bosentan described in the '740 patent involves the formation of undesired ethylene glycol bis-sulfonamide in which two molecules of the pyrimidine monohalide are coupled with one molecule of ethylene glycol. The removal of this impurity requires costly and laborious separation steps. This impurity is characterized as 1,2-bis[[5-(2-methoxyphenoxy)-2-pyrimidin-2yl-primidin-4yl]-4-tert-butyl-benzenesulfonamide]ethanediol (hereinafter referred to as the ‘bosentan dimer impurity’), which has the following structural formula:

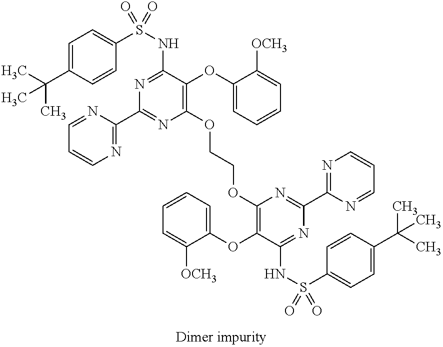

and is detected and resolved from bosentan by HPLC with an RRt of 1.77.

In a specific run, we have found that bosentan prepared by the above prior art procedures contained about above 0.5% and up to 5% of the dimer impurity before purification of the product at about 1.77 Relative Retention Time (RRt) measured by High Performance Liquid Chromatography (HPLC). The present inventors conducted experiments to purify the bosentan prepared by the above prior art procedures and found that the content of dimer impurity could be further reduced up to 0.15% by using the above mentioned re-crystallization procedures described in the prior art, and which could not be reduced to below 0.15% or eliminated completely.

Therefore, there remains a need for highly pure bosentan or a pharmaceutically acceptable salt thereof substantially free of the impurities, preferably deshydroxyethyl bosentan impurity and bosentan dimer impurity, as well as processes for preparing thereof.

Specific surface area of an active pharmaceutical ingredient may be affected by various factors. There is a general connection between Specific Surface Area and Particle Size; the smaller the Particle Size, the higher the Specific Surface Area. The rate of dissolution of a poorly-soluble drug is a rate-limiting factor in its absorption by the body. A reduction in the particle size can increase the dissolution rate of such compounds through an increase in the surface area of the solid phase that is in contact with the liquid medium, thereby resulting in an enhanced bioavailability of the compositions containing such compounds. It is generally not possible to predict the exact particle size and distribution required for any particular drug substance to achieve a specific dissolution profile or a specific in vivo behavior, as different drugs show differing dissolution characteristics with a reduction in the particle size.

Bosentan is a white to yellowish powder, poorly soluble in water (1.0 mg/100 ml) and in aqueous solutions at low pH (0.1 mg/100 ml at pH 1.1 and 4.0; 0.2 mg/100 ml at pH 5.0). The lack of solubility of bosentan creates a problem since bioavailability of a water insoluble active ingredient is usually poor. There is a need in the art to prepare active pharmaceutical ingredients such as bosentan particles with a desired surface area to obtain formulations with greater bioavailability, and to compensate for any loss of surface area before formulation.

There is a need in the art for highly pure bosentan or a pharmaceutically acceptable salt thereof substantially free of deshydroxyethyl bosentan impurity, with reduced particle size distribution, which has good flow properties, and better dissolution and solubility properties to obtain formulations with greater bioavailability.

### SUMMARY OF THE INVENTION

We have now surprisingly and unexpectedly found a novel crystalline form of bosentan, designated as crystalline form A₅, with high purity, adequate stability and good dissolution properties.

It has been surprisingly and unexpectedly found that the quantity of solvents used for isolation plays a critical role in obtaining the novel crystalline form A₅ of bosentan.

The novel crystalline form A₅ of bosentan is consistently reproducible, does not have the tendency to convert to other forms and found to be more stable even after being stored at a temperature of about 40° C. at a relative humidity of about 75% for at least about 1 month, specifically for a period of 6 months, or at a temperature of about 25° C. at a relative humidity of about 60% for at least about 6 months. Moreover, the crystalline form A₅ of bosentan has a tapped density of greater than about 0.6 g/ml and less electrostatic than the prior art forms, and has good flow properties, and which is particularly suitable for bulk preparation and handling, and so, the bosentan crystalline form A₅ of the present invention is suitable for formulating bosentan.

In one aspect, provided herein is the bosentan crystalline form A₅, characterized by data selected from the group consisting of:
i) a powder X-ray diffraction pattern substantially in accordance with FIG. 1;
ii) a powder X-ray diffraction pattern having peaks at about 7.15, 8.31, 9.26, 13.19, 18.63, 20.28 and 21.52±0.2 degrees 2-theta substantially as depicted in FIG. 1;
iii) a powder X-ray diffraction pattern having additional peaks at about 10.62, 11.32, 13.76, 14.33, 14.73, 15.23, 15.50, 16.10, 16.69, 17.75, 19.06, 22.68, 23.68, 24.41, 24.88, 25.77, 26.58, 27.37, 27.99, 29.01, 30.79, 31.24, 33.08 and 35.85±0.2 degrees 2-theta substantially as depicted in FIG. 1;
iv) an IR spectrum substantially in accordance with FIG. 2;
v) an IR spectrum having absorption bands at about 752, 997, 1020, 1083, 1112, 1203, 1252, 1292, 1453, 1579, 1926, 2962, 3064 and 3629±1 cm⁻¹;
vi) a DSC thermogram substantially in accordance with FIG. 3;
vii) a DSC thermogram having an endotherm peak in the range between about 120° C. and about 130° C. substantially as depicted in FIG. 3;
viii) a TGA thermogram substantially in accordance with FIG. 4; and
ix) a weight loss of about 3.0% to about 4.0% at a temperature of about 68° C. to about 100° C. as measured by TGA.

In one aspect, encompassed herein is a process for preparing the substantially pure and stable crystalline form A₅ of Bosentan.

In another aspect, the bosentan crystalline form A₅ has a water content of about 3.0-4.0% by weight, specifically about 3.0-3.8% by weight, and more specifically about 3.0-3.3% by weight, based on the total weight of the bosentan crystalline form A₅.

In an embodiment, the bosentan crystalline form A₅ of the present invention remains in the same crystalline form and stable, when stored at a temperature of about 25±2° C. and at a relative humidity of about 60±5% for a period of at least one month.

In another embodiment, the bosentan crystalline form A₅ of the present invention remains in the same crystalline form and stable, when stored at a temperature of about 25±2° C. and at a relative humidity of about 60±5% for a period of 6 months.

In yet another embodiment, the bosentan crystalline form A₅ of the present invention remains in the same crystalline form and stable, when stored at a temperature of about 40±2° C. and at a relative humidity of about 75±5% for a period of at least one month.

In still another embodiment, the bosentan crystalline form A₅ of the present invention remains in the same crystalline form and stable, when stored at a temperature of about 40±2° C. and at a relative humidity of about 75±5% for a period of 6 months.

In another aspect, provided herein is a pharmaceutical composition comprising crystalline form A₅ of Bosentan and one or more pharmaceutically acceptable excipients.

In still another aspect, provided herein is a pharmaceutical composition comprising crystalline form A₅ of Bosentan made by the process disclosed herein, and one or more pharmaceutically acceptable excipients.

In still further aspect, encompassed is a process for preparing a pharmaceutical formulation comprising combining crystalline form A₅ of Bosentan with one or more pharmaceutically acceptable excipients.

In another aspect, the crystalline form A₅ of Bosentan disclosed herein for use in the pharmaceutical compositions has a 90 volume-percent of the particles (D₉₀) having a size of less than or equal to about 400 microns, specifically less than or equal to about 300 microns, more specifically less than or equal to about 200 microns, still more specifically less than or equal to about 100 microns, and most specifically less than or equal to about 15 microns.

In another aspect, provided herein is an impurity of bosentan, p-tert-butyl-N-[6-hydroxy-5-(2-methoxyphenoxy)-2-(2-pyrimidinyl)-4-pyrimidinyl]benzenesulfonamide, designated as, ‘deshydroxyethyl bosentan impurity’, having the following structural formula I:

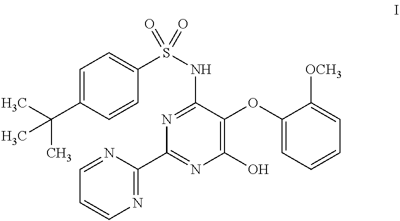

In another aspect, encompassed herein is a process for synthesizing and isolating the deshydroxyethyl bosentan impurity

In still another aspect, provided herein is a highly pure bosentan or a pharmaceutically acceptable salt thereof substantially free of deshydroxyethyl bosentan and bosentan dimer impurities.

As used herein, “highly pure bosentan or a pharmaceutically acceptable salt thereof substantially free of deshydroxyethyl bosentan and bosentan dimer impurities” refers to bosentan or a pharmaceutically acceptable salt thereof, in which bosentan has a purity of about 99% to about 99.99% and further comprising deshydroxyethyl bosentan and bosentan dimer impurities, each one, in an amount of less than about 0.15% as measured by HPLC. Specifically, the bosentan, as disclosed herein, contains less than about 0.1%, more specifically less than about 0.05%, still more specifically less than about 0.02% of each one of the deshydroxyethyl bosentan and bosentan dimer impurities, and most specifically essentially free of each one of the deshydroxyethyl bosentan and bosentan dimer impurities.

In another aspect, provided herein is bosentan or a pharmaceutically acceptable salt thereof comprising deshydroxyethyl bosentan impurity in an amount of about 0.01% to about 0.15%, specifically in an amount of about 0.01% to about 0.05%, as measured by HPLC.

In another aspect, the bosentan, obtained by the purification process as disclosed herein, contains less than about 0.1%, more specifically less than about 0.05%, still more specifically less than 0.02% of bosentan dimer impurity, and most specifically essentially free of bosentan dimer impurity.

In another aspect, provided herein is bosentan or a pharmaceutically acceptable salt thereof having purity of greater than about 99%, specifically greater than about 99.5%, more specifically greater than about 99.9%, and most specifically greater than about 99.95% as measured by HPLC.

In still further aspect, encompassed herein is a process for preparing the highly pure bosentan or a pharmaceutically acceptable salt thereof substantially free of deshydroxyethyl bosentan and bosentan dimer impurities.

In another aspect, provided herein is a pharmaceutical composition comprising highly pure bosentan or a pharmaceutically acceptable salt thereof substantially free of deshydroxyethyl bosentan and bosentan dimer impurities, and one or more pharmaceutically acceptable excipients.

In still another aspect, provided herein is a pharmaceutical composition comprising highly pure bosentan or a pharmaceutically acceptable salt thereof substantially free of deshydroxyethyl bosentan and bosentan dimer impurities made by the process disclosed herein, and one or more pharmaceutically acceptable excipients.

In still further aspect, encompassed is a process for preparing a pharmaceutical formulation comprising combining highly pure bosentan or a pharmaceutically acceptable salt thereof substantially free of deshydroxyethyl bosentan and bosentan dimer impurities with one or more pharmaceutically acceptable excipients.

In another aspect, the highly pure bosentan or a pharmaceutically acceptable salt thereof substantially free of substantially free of deshydroxyethyl bosentan and bosentan dimer impurities disclosed herein for use in the pharmaceutical compositions has a 90 volume-percent of the particles (D₉₀) having a size of less than or equal to about 300 microns, specifically less than or equal to about 200 microns, more specifically less than or equal to about 100 microns, still more specifically less than or equal to about 60 microns, and most specifically less than or equal to about 15 microns.

The highly pure bosetan and the crystalline form A₅ of bosentan disclosed herein may be used in the treatment of scleroderma and cardiovascular disorders such as ischemia, vasospasms and angina pectoris and hypertension (for example pulmonary hypertension).

Unless otherwise indicated, the following definitions are set forth to illustrate and define the meaning and scope of the various terms used to describe the invention herein.

The term “crystalline polymorph” refers to a crystal modification that can be characterized by analytical methods such as X-ray powder diffraction, IR-spectroscopy, differential scanning calorimetry (DSC) or by its melting point.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The expression “pharmaceutically acceptable salt” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use. Representative alkali or alkaline earth metal salts include the sodium, calcium, potassium and magnesium salts, and the like.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

As used herein, D_{X} means that X percent of the particles have a diameter less than a specified diameter D. Thus, a D₉₀ or d(0.9) of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

The term “water content” refers to the content of water based upon the Loss on Drying method as described in Pharmacopeial Forum, Vol. 24, No. 1, page 5438 (January-February 1998), the Karl Fisher assay for determining water content or thermogravimetric analysis (TGA). The calculation of water content is based upon the percent of weight that is lost by drying.

As used herein, the term, “detectable” refers to a measurable quantity measured using an HPLC method having a detection limit of 0.01 area-%.

As used herein, in connection with amount of impurities in bosentan or a pharmaceutically acceptable salt thereof, the term “not detectable” means not detected by the herein described HPLC method having a detection limit for impurities of 0.01 area-%.

### BRIEF DESCRIPTION OF THE DRAWINGS

FIG. 1 is a characteristic powder X-ray diffraction (XRD) pattern of Bosentan crystalline form A₅.

FIG. 2 is a characteristic Infrared (IR) spectrum of Bosentan crystalline form A₅.

FIG. 3 is a characteristic Differential Scanning Calorimetric (DSC) thermogram of Bosentan crystalline form A₅.

FIG. 4 is a characteristic Thermogravimetric Analysis (TGA) thermogram of Bosentan crystalline form A₅.

### DETAILED DESCRIPTION OF THE INVENTION

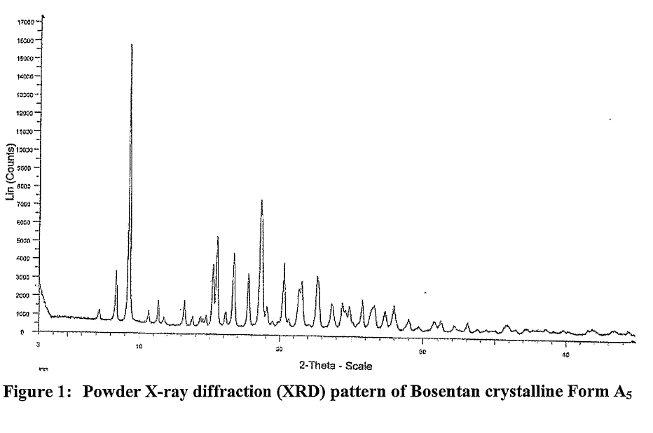

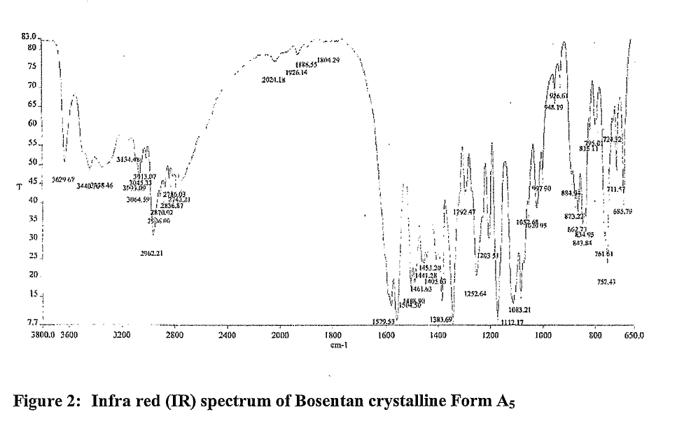

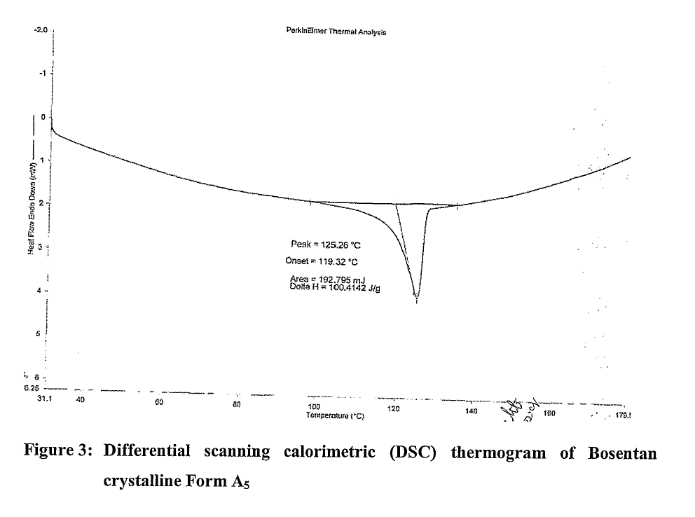

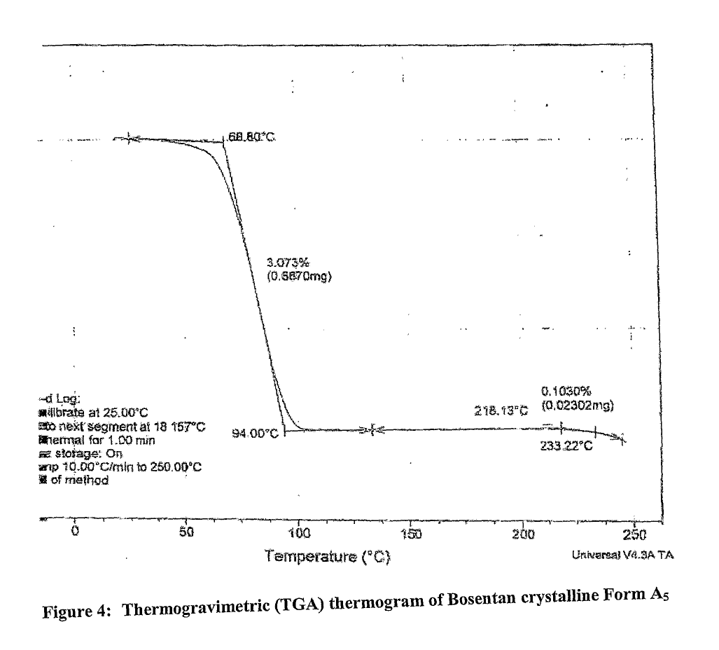

According to one aspect of the present invention, there is provided a novel crystalline form of Bosentan, designated as crystalline form A₅, characterized by data selected from the group consisting of:
i) a powder X-ray diffraction pattern substantially in accordance with FIG. 1;
ii) a powder X-ray diffraction pattern having peaks at about 7.15, 8.31, 9.26, 13.19, 18.63, 20.28 and 21.52±0.2 degrees 2-theta substantially as depicted in FIG. 1;
iii) a powder X-ray diffraction pattern having additional peaks at about 10.62, 11.32, 13.76, 14.33, 14.73, 15.23, 15.50, 16.10, 16.69, 17.75, 19.06, 22.68, 23.68, 24.41, 24.88, 25.77, 26.58, 27.37, 27.99, 29.01, 30.79, 31.24, 33.08 and 35.85±0.2 degrees 2-theta substantially as depicted in FIG. 1;
iv) an IR spectrum substantially in accordance with FIG. 2;
v) an IR spectrum having absorption bands at about 752, 997, 1020, 1083, 1112, 1203, 1252, 1292, 1453, 1579, 1926, 2962, 3064 and 3629±1 cm⁻¹;
vi) a DSC thermogram substantially in accordance with FIG. 3;
vii) a DSC thermogram having an endotherm peak in the range between about 120° C. and about 130° C. substantially as depicted in FIG. 3;
viii) a TGA thermogram substantially in accordance with FIG. 4; and
ix) a weight loss of about 3.0% to about 4.0% at a temperature of about 68° C. to about 100° C. as measured by TGA.

The measured weight loss of about 3.0% to about 4.0% indicates crystalline Form A₅ of Bosentan may be considered to be monohydrate by those skilled in the art.

According to another aspect, a process is provided for the preparation of crystalline form A₅ of bosentan, comprising:
a) forming a solution of bosentan in a first or second organic solvent in an amount of greater than about 6 ml per gram of bosentan, wherein the first organic solvent is an alcohol, a ketone, a nitrile, or a mixture thereof, and wherein the second organic solvent is a solvent medium comprising an alcohol and an ester solvent;
b) optionally, filtering the solvent solution to remove any extraneous matter; and
c) isolating crystalline form A₅ of bosentan from the solution.

The process can produce crystalline form A₅ of bosentan in substantially pure form.

The term “substantially pure bosentan crystalline form A₅” refers to the bosentan crystalline form A₅ having purity greater than about 99%, specifically greater than about 99.5%, more specifically greater than about 99.8% and still more specifically greater than about 99.9% (measured by HPLC).

In a preferred embodiment, the bosentan crystalline form A₅ has a water content of about 3.0-4.0% by weight, specifically about 3.0-3.8% by weight, and more specifically about 3.0-3.3% by weight, based on the total weight of the bosentan crystalline form A₅.

In another embodiment, the pure bosentan crystalline form A₅ obtained by above process has a water content of about 3.0-4.0% by weight, which is stable and consistently reproducible, and the moisture could not be removed even after extended drying for 12 hours at about 65° C. under vacuum.

The bosentan crystalline form A₅ obtained by the process disclosed herein is stable, consistently reproducible and has good flow properties, and which is particularly suitable for bulk preparation and handling, and so, the bosentan crystalline form A₅ obtained by the process disclosed herein is suitable for formulating bosentan.

In another embodiment, the bosentan crystalline form A₅ of the present invention remains in the same crystalline faun and stable even after being subjected to a mechanical process of reducing the size of particles which includes any one or more of cutting, chipping, crushing, milling, grinding, micronizing, or other particle size reduction methods known in the art.

In another embodiment, the bosentan crystalline form A₅ of the present invention remains in the same crystalline form and stable after being compressed under a pressure of about 7.5 tons/cm² for 10 to 15 minutes, as checked by X-ray diffractometer.

In another embodiment, the bosentan crystalline form A₅ of the present invention remains in the same crystalline form and stable, when stored at a temperature of about 25±2° C. and at a relative humidity of about 60±5% for a period of at least one month.

In still another embodiment, the bosentan crystalline form A₅ of the present invention remains in the same crystalline form and stable, when stored at a temperature of about 25±2° C. and at a relative humidity of about 60±5% for a period of 6 months.

In yet another embodiment, the bosentan crystalline form A₅ of the present invention remains in the same crystalline form and stable, when stored at a temperature of about 40±2° C. and at a relative humidity of about 75±5% for a period of at least one month.

In still another embodiment, the bosentan crystalline form A₅ of the present invention remains in the same crystalline form and stable, when stored at a temperature of about 40±2° C. and at a relative humidity of about 75±5% for a period of 6 months.

The term “remains stable”, as defined herein, refers to lack of formation of impurities, while being stored as described herein. The stability of crystalline form A₅ is measured by maintaining crystalline form A₅ at a temperature of about 40° C. at a relative humidity of about 75% for at least about 1 month, specifically for a period of 6 months, or at a temperature of about 25° C. at a relative humidity of about 60% for at least about 6 months.

The crystalline form A₅ of bosentan is a free-flowing solid, having a tapped density of at least about 0.5 g/ml, and specifically about 0.60 g/ml to about 0.75 g/ml.

Exemplary alcohol solvents include, but are not limited to, C₁ to C₄ straight or branched chain alcohol solvents such as methanol, ethanol, n-propanol, isopropanol, n-butanol, isobutanol, tert-butanol, and mixtures thereof. Specific alcohol solvents are methanol, ethanol, isopropanol, and mixtures thereof. Exemplary ketone solvents include, but are not limited to, acetone, methyl ethyl ketone, methyl isobutyl ketone, methyl tert-butyl ketone and the like, and mixtures thereof. A specific ketone solvent is acetone. Exemplary nitrile solvents include, but are not limited to, acetonitrile, propionitrile and the like, and mixtures thereof. A specific nitrile solvent is acetonitrile. Exemplary ester solvents include, but are not limited to, methyl acetate, ethyl acetate, isopropyl acetate, tert-butyl acetate, ethyl formate, and mixtures thereof. A specific ester solvent is ethyl acetate.

Specifically, the first organic solvent used in step-(a) is selected from the group consisting of methanol, ethanol, isopropanol, acetone, acetonitrile, and mixtures thereof, and more specifically methanol, ethanol, acetone, and mixtures thereof. Specifically the second organic solvent used in step-(a) is a solvent medium comprising an alcohol and ethyl acetate, and more specifically a solvent medium comprising methanol and ethyl acetate.

In one embodiment, the first or second organic solvent in an amount of about 6.2 ml to about 20 ml per gram of bosentan is used, specifically about 6.4 ml to about 15 ml per gram of bosentan is used, and most specifically about 6.5 ml to about 10.5 ml per gram of bosentan is used.

Step-(a) of forming a solution of bosentan includes dissolving any form of bosentan in the first or second organic solvent, or obtaining an existing solution from a previous processing step.

In one embodiment, the bosentan is dissolved in the first or second organic solvent at a temperature of below about reflux temperature of the solvent or solvent medium used, more specifically at about 30° C. to about 110° C., and still more specifically at about 50° C. to about 80° C.

As used herein, “reflux temperature” means the temperature at which the solvent or solvent system refluxes or boils at atmospheric pressure.

In another embodiment, the solution in step-(a) may be prepared by reacting 4-t-butyl-N-[6-chloro-5-(2-methoxyphenoxy)-2-(2-pyrimidinyl)-4-pyrimidinyl]benzene-sulfonamide with ethylene glycol in the presence of a suitable base, optionally in the presence of a phase transfer catalyst, in a suitable solvent under suitable conditions to produce a reaction mass containing crude bosentan, followed by usual work up such as washings, extractions, evaporations etc., and dissolving the resulting crude bosentan in the first or second organic solvent at a temperature of below reflux temperature of the solvent or solvent medium used, more specifically at about 30° C. to about 110° C., and still more specifically at about 50° C. to about 80° C.

In still another embodiment, the solution in step-(a) may be prepared by treating a pharmaceutically acceptable salt of bosentan with an acid to liberate bosentan and dissolving the bosentan in the first or second organic solvent.

Specific pharmaceutically acceptable salts of bosentan are obtained from alkali or alkaline earth metals include the sodium, calcium, potassium and magnesium, and more preferable salt being bosentan sodium.

The treatment of the pharmaceutically acceptable salt of bosentan with acid is carried out in any solvent and the selection of solvent is not critical. A wide variety of solvents such as chlorinated solvents, hydrocarbon solvents, ethers, alcohols, ketones, esters etc., can be used.

The acid can be inorganic or organic. Specific acids are hydrochloric acid, hydrobromic acid, sulfuric acid, nitric acid, acetic acid, oxalic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

The solution obtained in step-(a) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing bosentan by removing charcoal. In one embodiment, finely powdered carbon is an active carbon.

The solution obtained in step-(a) or step-(b) is optionally stirred at a temperature of about 30° C. to the reflux temperature of the solvent or solvent medium used for at least 20 minutes, and specifically at a temperature of about 40° C. to about 70° C. from about 30 minutes to about 5 hours.

The isolation of pure crystalline form A₅ of Bosentan in step-(c) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by combining an anti-solvent with the solution or a combination thereof.

In one embodiment, the crystallization is carried out by cooling the solution under stirring at a temperature of below 30° C. for at least 30 minutes, specifically at about 0° C. to about 30° C. from about 1 hour to about 20 hours, and more specifically at about 15° C. to about 25° C. from about 2 hours to about 18 hours.

In another embodiment, the crystallization is carried out by combining an anti-solvent with the solution followed by recovering the crystalline form A₅ of bosentan.

Exemplary anti-solvents include, but are not limited to, water; and ether solvents such as diisopropyl ether, diethyl ether, tetrahydrofuran, dioxane, and the like, and mixtures thereof. A specific anti-solvent is water.

The term “Anti-solvent” refers to a solvent which when added to an existing solution of a substance reduces the solubility of the substance.

The combining of the solution with anti-solvent is done in a suitable order, for example, the solution is added to the anti-solvent, or alternatively, the anti-solvent is added to the solution. The addition is carried out drop wise, in one portion, or in more than one portion. In one embodiment, addition is carried out at a temperature of below about 110° C. for at least 15 minutes, and more specifically at a temperature of about 40° C. to about 70° C. from about 20 minutes to about 2 hours. After completion of addition process, the resulting mass is stirred for at least 20 minutes, more specifically about 30 minutes to about 4 hours, at a temperature of about 20° C. to about 30° C.

Usually, about 0.5 to 3.0 volumes, specifically, about 0.9 to 1.2 volumes of anti-solvent with respect to the first or second organic solvent is used.

The pure crystalline form A₅ of Bosentan obtained may be recovered by conventional techniques known in the art such as filtration, filtration under vacuum, decantation, and centrifugation, or a combination thereof. In one embodiment, bosentan crystalline Form A₅ can be isolated by filtration employing a filtration media of, for example, a silica gel or celite.

The pure bosentan crystalline Form A₅ obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 65° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

Bosentan or a pharmaceutically acceptable salt of bosentan used as starting materials in the above process may be obtained by processes described in the prior art, or by the processes disclosed hereinafter.

The purity of the bosentan crystalline Form A₅ obtained by the process disclosed herein is of greater than about 99%, specifically greater than about 99.5%, more specifically greater than about 99.9%, and most specifically greater than about 99.95% as measured by HPLC. For example, the purity of the bosentan crystalline Form A₅ of the present invention can be about 99% to about 99.95%, or about 99.5% to about 99.99%.

Further encompassed herein is the use of bosentan crystalline Form A₅ for the manufacture of a pharmaceutical composition.

A specific pharmaceutical composition of bosentan crystalline Form A₅ is selected from a solid dosage form and an oral suspension.

In one embodiment, the bosentan crystalline Form A₅ of the present invention has a D₉₀ particle size of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the substantially pure bosentan crystalline Form A₅ disclosed herein for use in the pharmaceutical compositions has a 90 volume-percent of the particles (D₉₀) have a size of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the particle sizes of bosentan crystalline Form A₅ can be achieved by a mechanical process of reducing the size of particles which includes any one or more of cutting, chipping, crushing, milling, grinding, micronizing, trituration or other particle size reduction methods known in the art, to bring the solid state forms the desired particle size range.

According to another aspect of the present invention, there is provided an impurity of bosentan, deshydroxyethyl bosentan, p-tert-butyl-N-[6-hydroxy-5-(2-methoxyphenoxy)-2-(2-pyrimidinyl)-4-pyrimidinyl]benzenesulfonamide, having the following structural formula I:

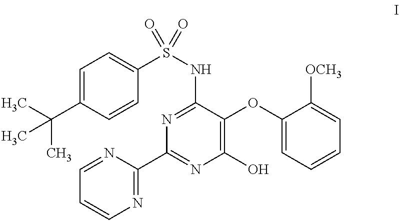

According to another aspect of the present invention, there is provided a process for synthesizing and isolating the deshydroxyethyl bosentan impurity of formula I comprising reacting p-tert-Butyl-N-[6-chloro-5-(2-methoxyphenoxy)-2-(2-pyrimidinyl)-4-pyrimidinyl]benzenesulfonamide (chloro compound) with a suitable base in a solvent or a mixture of solvents at elevated temperature to produce a reaction mass, and isolating the deshydroxyethyl bosentan as a solid.

Preferable solvents are those that dissolve the chloro compound to ensure maximum contact between the reactants resulting in faster reaction. However, the process is also operable with solvents that only partially dissolve the chloro compound. Specific solvents are toluene, ethylene glycol, xylene, tetrahydrofuran, dimethylformamide, diphenyl ether and mixtures thereof, and more preferable solvent is diphenyl ether.

The suitable base is a strong alkali, selected from the group consisting of hydroxides of alkali metals. Specific bases are sodium hydroxide and potassium hydroxide.

In an embodiment, the reaction is carried out at a temperature of about 50° C. to the reflux temperature of the solvent used, specifically at a temperature of about 80° C. to the reflux temperature of the solvent used, more specifically at a temperature of about 100° C. to the reflux temperature of the solvent used, and most specifically at the reflux temperature of the solvent used.

Time required for completion of the reaction depends on factors such as solvent used and temperature at which the reaction is carried out. For example, if the reaction is carried out in diphenyl ether under reflux conditions, from about 15 minutes to 5 hours is required for the reaction completion.

Usually, about 1 to 15 moles, preferably, about 11 moles of base per 1 mole of chloro compound is used.

The reaction mass containing the deshydroxyethyl bosentan obtained is optionally treated with an acid, for example hydrochloric acid, followed by usual work up such as washings, extractions etc, and then isolated as a solid from a suitable solvent by conventional methods such as cooling, seeding, partial removal of the solvent from the solution, by adding an anti-solvent to the solution, evaporation, vacuum drying, spray drying, freeze drying, or a combination thereof.

The solvent used for isolating the deshydroxyethyl bosentan is selected from the group consisting of water, acetone, methanol, ethanol, n-propanol, isopropanol, ethyl acetate, dichloromethane, n-pentane, n-hexane, n-heptane, cyclohexane, toluene, and mixtures thereof.

According to another aspect of the present invention, there is provided a highly pure bosentan or a pharmaceutically acceptable salt thereof substantially free of deshydroxyethyl bosentan and bosentan dimer impurities.

As used herein, “highly pure bosentan or a pharmaceutically acceptable salt thereof substantially free of deshydroxyethyl bosentan and bosentan dimer impurities” refers to bosentan or a pharmaceutically acceptable salt thereof, in which bosentan has a purity of about 99% to about 99.99% and further comprising deshydroxyethyl bosentan and bosentan dimer impurities, each one, in an amount of less than about 0.15% as measured by HPLC. Specifically, the bosentan, as disclosed herein, contains less than about 0.1%, more specifically less than about 0.05%, still more specifically less than about 0.02% of each one of the deshydroxyethyl bosentan and bosentan dimer impurities, and most specifically essentially free of each one of the deshydroxyethyl bosentan and bosentan dimer impurities.

In a preferred embodiment, the highly pure bosentan or a pharmaceutically acceptable salt thereof of the present invention comprises deshydroxyethyl bosentan impurity in an amount of about 0.01% to about 0.15%, specifically in an amount of about 0.01% to about 0.05%, as measured by HPLC.

In another embodiment, the highly pure bosentan, as disclosed herein, contains less than about 0.1%, more specifically less than about 0.05%, still more specifically less than 0.02% of bosentan dimer impurity, and most specifically essentially free of bosentan dimer impurity.

The term “bosentan or a pharmaceutically acceptable salt thereof essentially free of deshydroxyethyl bosentan impurity” refers to bosentan or a pharmaceutically acceptable salt thereof contains a non-detectable amount of deshydroxyethyl bosentan impurity.

The term “bosentan or a pharmaceutically acceptable salt thereof essentially free of bosentan dimer impurity” refers to bosentan or a pharmaceutically acceptable salt thereof contains a non-detectable amount of bosentan dimer impurity.

Preferable pharmaceutically acceptable salts of bosentan are obtained from alkali or alkaline earth metals include the sodium, calcium, potassium and magnesium, and more preferable salt being bosentan sodium.

The highly pure bosentan or a pharmaceutically acceptable salt thereof of the present invention has a purity of greater than about 99%, specifically greater than about 99.5%, more specifically greater than about 99.9%, and most specifically greater than about 99.95% as measured by HPLC. For example, the purity of the highly pure bosentan of the present invention can be about 99% to about 99.95%, or about 99.5% to about 99.99%.

According to another aspect of the present invention, a process is provided for the preparation of highly pure bosentan or a pharmaceutically acceptable salt thereof substantially free of deshydroxyethyl bosentan and bosentan dimer impurities, comprising:
a) forming a solution of crude bosentan in a solvent medium comprising ethyl acetate and an alcohol solvent;
b) optionally, filtering the solvent solution to remove any extraneous matter; and
c) isolating highly pure bosentan substantially free of deshydroxyethyl bosentan and bosentan dimer impurities from the solution, and optionally converting the highly pure bosentan obtained into its pharmaceutically acceptable salts thereof.

The term ‘crude bosentan or a pharmaceutically acceptable salt thereof’ in the specification refers to bosentan or a pharmaceutically acceptable salt thereof containing at least one, or both, of the deshydroxyethyl bosentan and bosentan dimer impurities, each one in an amount of greater than 0.15% as measured by HPLC.

Exemplary alcohol solvents used in step-(a) include, but are not limited to, Cᵢ to C₅ straight or branched chain alcohol solvents such as methanol, ethanol, n-propanol, isopropanol, n-butanol, isobutanol, tert-butanol, amyl alcohol, isoamyl alcohol, and mixtures thereof. Specific alcohol solvents are methanol, ethanol, isopropyl alcohol, and mixtures thereof, and more specifically methanol.

Usually, about 0.5 to 6.0 volumes, specifically, about 2.0 to 3.0 volumes of the alcohol solvent with respect to ethyl acetate can be used.

Step-(a) of forming a solution of crude bosentan includes dissolving any form of bosentan in the solvent medium, or obtaining an existing solution from a previous processing step.

In one embodiment, the bosentan is dissolved in the solvent medium at a temperature of about 30° C. to the reflux temperature of the solvent medium used, more specifically at about 40° C. to about 80° C., and still more specifically at about 50° C. to about 70° C.

As used herein, “reflux temperature” means the temperature at which the solvent or solvent system refluxes or boils at atmospheric pressure.

In another embodiment, the solution in step-(a) is prepared by reacting 4-t-butyl-N-[6-chloro-5-(2-methoxyphenoxy)-2-(2-pyrimidinyl)-4-pyrimidinyl]benzenesulfonamide with ethylene glycol in the presence of a suitable base, optionally in the presence of a phase transfer catalyst, in a suitable solvent under suitable conditions to produce a reaction mass containing crude bosentan, followed by usual work up such as washings, extractions, evaporations etc., and dissolving the resulting crude bosentan in the solvent medium at a temperature of about 30° C. to the reflux temperature of the solvent medium used, more specifically at about 40° C. to about 80° C., and still more specifically at about 50° C. to about 70° C.

The base used in the above reaction is selected from the group consisting of hydroxides and alkoxides of alkali or alkaline earth metals. Specifically, the base is selected from the group consisting of sodium hydroxide, calcium hydroxide, magnesium hydroxide, potassium hydroxide, lithium hydroxide, sodium tert-butoxide, sodium isopropoxide and potassium tert-butoxide; more specifically, the base is selected from the group consisting of sodium hydroxide, potassium hydroxide, calcium hydroxide, and magnesium hydroxide; and a most specific base is sodium hydroxide.

Specifically, the reaction is carried out at a temperature of about 0° C. to the reflux temperature of the solvent used, more specifically at about 40° C. to the reflux temperature of the solvent used, still more specifically at about 60° C. to the reflux temperature of the solvent used, and most specifically at the reflux temperature of the solvent used.

In still another embodiment, the solution in step-(a) may be prepared by treating a pharmaceutically acceptable salt of bosentan with an acid to liberate crude bosentan and dissolving the crude bosentan in the solvent medium.

Specific pharmaceutically acceptable salts of bosentan are obtained from alkali or alkaline earth metals include the sodium, calcium, potassium and magnesium, and more preferable salt being bosentan sodium.

The treatment of the pharmaceutically acceptable salt of bosentan with acid is carried out in any solvent and the selection of solvent is not critical. A wide variety of solvents such as chlorinated solvents, hydrocarbon solvents, ethers, alcohols, ketones, esters etc., can be used.

The acid can be inorganic or organic. Specific acids are hydrochloric acid, hydrobromic acid, sulfuric acid, nitric acid, acetic acid, oxalic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

The solution obtained in step-(a) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing bosentan by removing charcoal. In one embodiment, finely powdered carbon is an active carbon.

The solution obtained in step-(a) or step-(b) is optionally stirred at a temperature of about 30° C. to the reflux temperature of the solvent medium used for at least 20 minutes, and specifically at a temperature of about 40° C. to about 70° C. from about 30 minutes to about 5 hours.

The isolation of highly pure bosentan substantially free of deshydroxyethyl bosentan and bosentan dimer impurities in step-(c) is carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by combining an anti-solvent with the solution or a combination thereof. In one embodiment, the crystallization is carried out by cooling the solution at a temperature of below 30° C. for at least 30 minutes, specifically at about 0° C. to about 30° C. from about 1 hour to about 20 hours, and more specifically at about 15° C. to about 25° C. from about 2 hours to about 18 hours.

The highly pure bosentan substantially free of deshydroxyethyl bosentan and bosentan dimer impurities obtained may be recovered by conventional techniques known in the art such as filtration, filtration under vacuum, decantation, and centrifugation, or a combination thereof. In one embodiment, the highly pure bosentan can be isolated by filtration employing a filtration media of, for example, a silica gel or celite.

The highly pure bosentan obtained by the above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 65° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

Pharmaceutically acceptable salts of bosentan can be prepared in high purity by using the highly pure bosentan obtained by the method disclosed herein, by known methods.

Preferable pharmaceutically acceptable salts of bosentan are obtained from alkali or alkaline earth metals include the sodium, calcium, potassium and magnesium, and more preferable salt being bosentan sodium.

The purity of the bosentan obtained after purification process disclosed herein is of greater than about 99%, specifically greater than about 99.5%, more specifically greater than about 99.9%, and most specifically greater than about 99.95% as measured by HPLC. For example, the purity of the bosentan of the present invention can be about 99% to about 99.95%, or about 99.5% to about 99.99%.

According to another aspect of the present invention, there is provided highly pure bosentan substantially free of deshydroxyethyl bosentan and bosentan dimer impurities, has a relatively low content of one or more organic volatile impurities.

In an embodiment, the bosentan obtained by the purification process disclosed herein, is having less than about 1000 parts per million (ppm) methanol, less than about 3000 ppm acetone, less than about 300 ppm methylene chloride, less than about 3000 ppm ethyl acetate, less than about 300 ppm toluene, and less than about 150 ppm ethylene glycol, as measured by GC.

In another embodiment, the bosentan obtained by the purification process disclosed herein, is having less than about 120 parts per million (ppm) methanol, less than about 100 ppm acetone, less than about 10 ppm methylene chloride, less than about 100 ppm ethyl acetate, less than about 10 ppm toluene, and less than about 1 ppm ethylene glycol, as measured by GC.

In another embodiment, the bosentan, obtained by the purification process disclosed herein, is having less than about 150 ppm ethylene glycol, specifically less than about 50 ppm ethylene glycol, more specifically less than about 1 ppm ethylene glycol, and most specifically essentially free from ethylene glycol, as measured by GC.

More specifically, the bosentan obtained by the purification process disclosed herein is having the overall level of organic volatile impurities in an amount of less than about 1500 ppm, more specifically less than about 500 ppm, and most specifically less than about 150 ppm.

Further encompassed herein is the use of highly pure bosentan or a pharmaceutically acceptable salt thereof substantially free of deshydroxyethyl bosentan and bosentan dimer impurities for the manufacture of a pharmaceutical composition.

A specific pharmaceutical composition of highly pure bosentan or a pharmaceutically acceptable salt thereof substantially free of deshydroxyethyl bosentan and bosentan dimer impurities is selected from a solid dosage form and an oral suspension.

In one embodiment, the highly pure bosentan or a pharmaceutically acceptable salt thereof of the present invention has a D₉₀ particle size of less than or equal to about 300 microns, specifically less than or equal to about 200 microns, more specifically less than or equal to about 100 microns, still more specifically less than or equal to about 60 microns, and most specifically less than or equal to about 15 microns.

In another embodiment, the highly pure bosentan or a pharmaceutically acceptable salt thereof disclosed herein for use in the pharmaceutical compositions has a 90 volume-percent of the particles (D₉₀) have a size of less than or equal to about 300 microns, specifically less than or equal to about 200 microns, more specifically less than or equal to about 100 microns, still more specifically less than or equal to about 60 microns, and most specifically less than or equal to about 15 microns.

In another embodiment, the particle sizes of highly pure bosentan or a pharmaceutically acceptable salt thereof can be achieved by a mechanical process of reducing the size of particles which includes any one or more of cutting, chipping, crushing, milling, grinding, micronizing, trituration or other particle size reduction methods known in the art, to bring the solid state forms the desired particle size range.

According to another aspect, there is provided pharmaceutical compositions comprising bosentan crystalline Form A₅ prepared according to processes disclosed herein and one or more pharmaceutically acceptable excipients.

According to another aspect, there is provided a process for preparing a pharmaceutical formulation comprising combining bosentan crystalline Form A₅ prepared according to processes disclosed herein, with one or more pharmaceutically acceptable excipients.

According to another aspect, there is provided pharmaceutical compositions comprising highly pure bosentan or a pharmaceutically acceptable salt thereof substantially free of deshydroxyethyl bosentan and bosentan dimer impurities prepared according to processes disclosed herein and one or more pharmaceutically acceptable excipients.

According to another aspect, there is provided a process for preparing a pharmaceutical formulation comprising combining highly pure bosentan or a pharmaceutically acceptable salt thereof substantially free of deshydroxyethyl bosentan and bosentan dimer impurities prepared according to processes disclosed herein, with one or more pharmaceutically acceptable excipients.

Yet another embodiment, disclosed herein are pharmaceutical compositions comprising a therapeutically effective amount of bosentan crystalline Form A₅ or highly pure bosentan or a pharmaceutically acceptable salt thereof substantially free of deshydroxyethyl bosentan and bosentan dimer impurities obtained by the processes disclosed herein. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The bosentan crystalline Form A₅ or highly pure bosentan or a pharmaceutically acceptable salt thereof substantially free of deshydroxyethyl bosentan and bosentan dimer impurities obtained by the processes disclosed herein may also be administered as suppositories, ophthalmic ointments and suspensions, and parenteral suspensions, which are administered by other routes.

The dosage forms may contain bosentan crystalline Form A₅ or highly pure bosentan or a pharmaceutically acceptable salt thereof substantially free of deshydroxyethyl bosentan and bosentan dimer impurities obtained by the processes disclosed herein as is or, alternatively, may contain bosentan crystalline Form A₅ or highly pure bosentan or a pharmaceutically acceptable salt thereof substantially free of deshydroxyethyl bosentan and bosentan dimer impurities of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosages contain bosentan crystalline Form A₅ or highly pure bosentan or a pharmaceutically acceptable salt thereof substantially free of deshydroxyethyl bosentan and bosentan dimer impurities of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction:

The X-Ray powder diffraction was measured by an X-ray powder Diffractometer equipped with a Cu-anode (λ=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2θ; step width=0.01579°; and measuring time per step=0.11 second.

### Infra Red (FT-IR) Spectroscopy:

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 3800 to 650 cm⁻¹.

### Differential Scanning Calorimetry (DSC):

DSC (Differential Scanning Calorimetry) measurements were performed with a Differential Scanning Calorimeter (Diamond DSC, Perkin-Elmer) at a scan rate of 5° C. per minute. The nitrogen gas purge was at 40 ml/min. The instrument was calibrated for temperature and heat flow using indium as standards. The samples were encapsulated in to closed aluminium pans without hole subsequently crimped to ensure a tight seal. Data acquisition and analysis were performed using pyris software.

### Thermogravimetry (TGA):

Thermogravimetric analysis was performed with a TGA Q500 of TA instruments, Lukens-Drive, Delware, USA.

### High Performance Liquid Chromatography (HPLC):

The purity was measured by high performance liquid chromatography under the following conditions:
Apparatus: Water's HPLC system having alliance 2695 model pump and 2487 (UV) detector with Empower chromatography software or its equivalent.
Chromatographic Parameters:

Column: Zorbax SB-Phenyl 150×4.6 mm×3.5 μm

Detector: UV at 220 nm

Flow rate: 1.0 ml/min

Injection volume: 10.0 μL

Run time: 50 min

Column temperature: 30° C.

Sample temperature: Ambient

Diluent: Water:Acetonitrile-50:50 (% v/v)

### Gas Chromatography:

Instrument: Gas chromatograph equipped with FID detector and headspace. Instrument: Agilent 6890 plus gas chromatograph equipped with FID detector and Gerstel Headspace.
Column: Rtx-624, 75 m×0.53 mm ID, 3 μm
Column Temperature: 40° C. (hold for 10 minutes) to 240° C. at 20° C./minute, hold at 240° C. for 5 minutes.
Injector/detector: 250° C./300° C. Carrier gas: Nitrogen at 30 cm/sec, linear velocity
Split Ratio: (2:1)
Head Space Parameters:
Incubation Temperature: 95° C.
Incubation Time: 30 minutes
Agitation Speed: 600 rpm
Syringe Temperature: 115° C.
Injection Volume: 1 ml

The following examples are provided to enable one skilled in the art to practice the invention and are merely illustrate the process of this invention. However, it is not intended in any way to limit the scope of the present invention.

### Examples

### Example 1

### Preparation of Crude Bosentan

A mixture of 4-t-butyl-N-[6-chloro-5-(2-methoxyphenoxy)-2-(2-pyrimidinyl)-4-pyrimidinyl]benzenesulfonamide (5 g), ethylene glycol (50 ml) and sodium hydroxide (1.52 gm) was heated at 92-97° C. for 5 hours. The resulting brownish solution was allowed to cool at 70-80° C. and the resulting mass was added to water (25 ml). The resulted sticky mass was extracted two times with dichloromethane (2×50 ml). The organic extracts were taken up in water (50 ml) followed by the addition of tartaric acid solution (10 ml) to adjust pH of the mass to 1-2. The resulting organic layer was dried over sodium sulphate (5 gm) and then concentrated under reduced pressure. The oily residue was dissolved in a mixture of ethyl acetate (15 ml) and methanol (35 ml) to get a clear solution. The resulted solution was cooled at 25° C. over 2 hours and then stirred for 15 hours at 25-30° C. The resulted precipitate was filtered and then dried under vacuum at 55-60° C. to give 3.5 g of crude bosentan [Purity by HPLC: 99.11%; content of deshydroxyethyl bosentan impurity: 0.35%; content of dimer impurity: 0.2%].

### Example 2

### Preparation of Crude Bosentan

A mixture of 4-t-butyl-N-[6-chloro-5-(2-methoxyphenoxy)-2-(2-pyrimidinyl)-4-pyrimidinyl]benzenesulfonamide (10 g), ethylene glycol (100 ml) and sodium hydroxide (3.04 g) was heated at 92-97° C. for 5 hours. The resulted brown colored solution was allowed to cool at 70-80° C. and the resulting mass was added to water (50 ml). The resulted sticky mass was extracted two times with dichloromethane (2×100 ml). The organic extracts were taken up in water (100 ml) followed by the addition of tartaric acid solution (20 ml) to adjust pH of the mass to 1-2. The resulting organic layer was dried over sodium sulphate (10 gm) and followed by evaporation on rotavapour under reduced pressure. The semisolid residue was dissolved in ethanol (10 ml) at 70-75° C. and followed by the addition of water (10 ml) over 20 minutes. The resulted mass was cooled at 20° C. over 2 hours and then stirred for 18 hours at 20-25° C. The precipitated product was filtered under vacuum and then dried at 55-60° C. to get 8.5 gm of crude bosentan (Purity by HPLC: 98.55%; content of deshydroxyethyl bosentan impurity: 0.45%; content of dimer impurity: 0.25%).

### Example 3

### Preparation of Crystalline Form A₅ of Bosentan

Bosentan (30g) was taken in a mixture of methanol (210 ml) and ethyl acetate (90 ml) and the resulting mixture was heated at 55-65° C. for 10-15 minutes to form a clear solution. The resulting solution was cooled gradually at 20 to 30° C. and then stirred for 17-18 hours at 20-25° C. The resulting solid was filtered, washed with a mixture of methanol (21 ml) and ethyl acetate (9 ml) and then dried the material under vacuum at 60-65° C. to give 23.8 g of crystalline form A₅ of Bosentan (Purity by HPLC: 99.94%; Water Content: 3.07% by weight; Tapped density: 0.668 g/ml; content of deshydroxyethyl bosentan impurity: Not detected; content of dimer impurity: 0.03%).
Level of organic volatile impurities: Methanol—90 ppm; Acetone—Not detected; Methylene chloride—Not detected; Ethyl acetate—21 ppm; Toluene—Not detected; and Ethylene glycol—Not detected.

### Example 4

### Preparation of Crystalline Form A₅ of Bosentan

Bosentan (3 g) was taken in acetone (20 ml) and heated at 50-60° C. for 10-15 minutes to form a clear solution. This was followed by the addition of water (20 ml) at 60-65° C. and the resulting mixture was then cooled gradually at 20-25° C. The reaction mixture was further stirred at 20-25° C. for 18 hours. The resulting solid was filtered, washed with acetone (2 ml) and then dried the material under vacuum at 55-65° C. to yield 1.8 g of crystalline form A₅ of Bosentan (Purity by HPLC: 99.90%; Water Content: 3.5% by weight; Tapped density: 0.714 g/ml).

### Example 5

### Preparation of Crystalline Form A₅ of Bosentan

Bosentan (3 g) was taken in ethanol (20 ml) and heated at 50-65° C. for 10 minutes to form a clear solution. This was followed by the addition of water (20 ml) at 50-65° C. The reaction mass was then gradually cooled to 20-25° C. followed by stirring at 20-25° C. for 18 hours. The resulting solid was filtered, washed with ethanol (2 ml) and then dried the material under vacuum at 55-65° C. to yield 2.2 g of crystalline form A₅ of Bosentan (Purity by HPLC: 99.91%; Water Content: 3.2% by weight; Tapped density: 0.626 g/ml).

### Example 6

### Preparation of Crystalline Form A₅ of Bosentan

Bosentan (30 g) was taken in methanol (300 ml) and the resulting mixture was heated at 55-65° C. for 10-15 minutes to form a clear solution. The resulting solution was gradually cooled at 20-30° C. and then stirred for 18 hours at 20-25° C. The resulting solid was filtered, washed with methanol (30 ml) and then dried the material under vacuum at 55-65° C. to give 22.5 g of crystalline form A₅ of Bosentan (Purity by HPLC: 99.92%; Water Content: 3.8% by weight; Tapped density: 0.667 g/ml).

### Example 7

### Purification of Crude Bosentan

Crude bosentan (3 gm) was taken in a mixture of ethyl acetate (9 ml) and methanol (21 ml) and the resulting mixture was heated at 55-65° C. for 10-15 minutes to form a clear solution. The solution was cooled at 25° C. for 1 hour and then stirred for 18 hours at 20-25° C. The resulted precipitate was filtered and then dried under vacuum at 55-65° C. to yield 2.3 gm of pure bosentan (Purity by HPLC: 99.92%; content of deshydroxyethyl bosentan impurity: 0.03%; content of dimer impurity: 0.04%; Water content by KF: 3.2% by weight).
Particle size distribution: d(0.1)=13.39 microns, d(0.5) =85.61 microns, d(0.9)=231.09.
Level of organic volatile impurities: Methanol—110 ppm; Acetone—Not detected; Methylene chloride—Not detected; Ethyl acetate—43 ppm; Toluene—Not detected; and Ethylene glycol—Not detected.

### Example 8

Bosentan (obtained from Examples 3-7) was fine-milled by being passed through a grinder (Make: Morphy Richards, Model-Icon DLX) having stainless steel liquidizing blade for 3-4 minutes to where 90% of the bosentan particles had a diameter of less than about 60 microns.

### Example 9

Bosentan (obtained as per the processes described in Examples 3-7) was grinded in a mixer (Make: Morphy Richards, Model-Icon DLX) having stainless steel liquidizing blade for 3-4 minutes. The obtained powder was passed through a sieve (B.S.S.-100, A.S.T.M—100, Micron—150) to get 90% of the bosentan particles have a diameter of less than about 130 microns [Particle size distribution: d(0.1)=11.76 microns, d(0.5)=28.95 microns, d(0.9)=120.88 (Example 9A in Table 1]. The particle size distribution of 4 additional samples, obtained according to the procedure described in example 9, are detailed in Table 1 (Examples 9B, 9C, 9D and 9E).

TABLE 1
Particle Size Distribution

| Example | d(0.1) microns | d(0.5) microns | d(0.9) microns |
|---|---|---|---|
| 9A | 3.49 | 28.95 | 120.88 |
| 9B | 11.76 | 38.96 | 78.25 |
| 9C | 7.89 | 31.92 | 69.43 |
| 9D | 14.85 | 53.73 | 109.61 |
| 9E | 17.03 | 58.84 | 127.11 |

### Example 10

### Preparation of p-tert-Butyl-N-[6-hydroxy-5-(2-methoxyphenoxy)-2-(2-pyrimidinyl)-4-pyrimidinyl]benzenesulfonamide (Deshydroxyethyl bosentan impurity)

A mixture of p-tert-Butyl-N-[6-chloro-5-(2-methoxyphenoxy)-2-(2-pyrimidinyl)-4-pyrimidinyl]benzenesulfonamide (2 gm) and potassium hydroxide pellets (2.5 gm) was heated to 150° C. to form a suspension followed by the addition of diphenyl ether (10 ml) at 150° C. The reaction mass temperature was increased to 175° C. and stirred till completion of the reaction. The resulting mass was cooled to 25-30° C. followed by the addition of water (50 ml) and toluene (25 ml) at 25-30° C. The resulting two layers were separated and the aqueous layer was washed with toluene (50 ml) at 25-30° C. The aqueous layer was cooled to 15° C. followed by the addition of concentrated hydrochloric acid (1.5 ml) and adjusted the pH to 2 at 10-15° C. The resulted solid was filtered and washed with water. The white colored solid was dried at 50-55 ° C. to yield 1.2 gm of p-tert-Butyl-N-[6-hydroxy-5-(2-methoxyphenoxy)-2-(2-pyrimidinyl)-4-pyrimidinyl]benzenesulfonamide (HPLC Purity: 99.1%).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

We claim:

1. A crystalline form A₅ of Bosentan or a pharmaceutical composition comprising bosentan crystalline Form A₅, wherein the crystalline form A₅ of Bosentan, has a purity of about 99% to about 99.99% as measured by HPLC, is characterized by data selected from the group consisting of:
i) a powder X-ray diffraction pattern substantially in accordance with FIG. 1;
ii) a powder X-ray diffraction pattern having peaks at about 7.15, 8.31, 9.26, 13.19, 18.63, 20.28 and 21.52±0.2 degrees 2-theta;
iii) a powder X-ray diffraction pattern having additional peaks at about 10.62, 11.32, 13.76, 14.33, 14.73, 15.23, 15.50, 16.10, 16.69, 17.75, 19.06, 22.68, 23.68, 24.41, 24.88, 25.77, 26.58, 27.37, 27.99, 29.01, 30.79, 31.24, 33.08 and 35.85±0.2 degrees 2-theta;
iv) an IR spectrum substantially in accordance with FIG. 2;
v) an IR spectrum having absorption bands at about 752, 997, 1020, 1083, 1112, 1203, 1252, 1292, 1453, 1579, 1926, 2962, 3064 and 3629±1 cm⁻¹;
vi) a DSC thermogram substantially in accordance with FIG. 3;
vii) a DSC thermogram having an endotherm peak in the range between about 120° C. and about 130° C.;
viii) a TGA thermogram substantially in accordance with FIG. 4; and
ix) a weight loss of about 3.0% to about 4.0% at a temperature of about 68° C. to about 100° C. as measured by TGA.

2. The crystalline form of claim 1, having a water content of about 3.0-4.0% by weight, based on the total weight of the bosentan crystalline form A₅; and wherein the crystalline form A₅ of Bosentan has a tapped density of about 0.60 g/ml to about 0.75 g/ml.

3-9. (canceled)

10. A process for the preparation of bosentan crystalline form A₅ of claim 1, comprising:
a) forming a solution of bosentan in an organic solvent in an amount of greater than about 6 ml per gram of bosentan, wherein the organic solvent is methanol, ethanol, acetone, or a solvent medium comprising methanol and ethyl acetate;
b) optionally, filtering the solvent solution to remove any extraneous matter; and
c) isolating crystalline form A₅ of bosentan from the solution.

11-14. (canceled)

15. The process of claim 10, wherein the organic solvent is used in an amount of about 6.2 ml to about 20 ml per gram of bosentan.

16. The process of claim 15, wherein the organic solvent is used in an amount of about 6.5 ml to about 10.5 ml per gram of bosentan.

17. The process of claim 10, wherein the solution in step-(a) is formed either i) by dissolving bosentan in the specified amount of organic solvent at a temperature of below about reflux temperature of the solvent or solvent medium used; ii) by reacting 4-t-butyl-N-[6-chloro-5 -(2-methoxyphenoxy)-2-(2-pyrimidinyl)-4-pyrimidinyl]benzenesulfonamide with ethylene glycol in the presence of a suitable base, optionally in the presence of a phase transfer catalyst, in a suitable solvent under suitable conditions to produce a reaction mass containing crude bosentan, subjecting the reaction mass to washings, extractions or evaporations, and dissolving the resulting crude bosentan in the specified amount of organic solvent at a temperature of below reflux temperature of the solvent or solvent medium used; or iii) by treating a pharmaceutically acceptable salt of bosentan with an acid to liberate bosentan and dissolving the bosentan in the specified amount of organic solvent.

18. The process of claim 17, wherein the dissolution is carried out at a temperature of about 30° C. to about 110° C.

19-21. (canceled)

22. The process of claim 10, wherein the solution obtained in step-(a) is further subjected to carbon treatment; wherein the isolation of pure crystalline form A₅ of Bosentan in step-(c) is initiated by cooling, seeding, partial removal of the solvent from the solution, by combining an anti-solvent with the solution or a combination thereof; wherein the pure crystalline form A₅ of Bosentan obtained in step-(c) is recovered by filtration, filtration under vacuum, decantation, and centrifugation, or a combination thereof; and wherein the pure crystalline form A₅ of Bosentan obtained in step-(c) is further dried under vacuum or at atmospheric pressure, at a temperature of about 35° C. to about 65° C.

23-24. (canceled)

25. The process of claim 22, wherein the isolation is carried out either by cooling the solution under stirring at a temperature of below 30° C. for at least 30 minutes, or by combining the solution with an anti-solvent wherein the anti-solvent is water.

26. The process of claim 25, wherein the isolation is carried out by cooling the solution under stirring at a temperature of about 0° C. to about 30° C. for about 1 hour to about 20 hours.

27-44. (canceled)

45. Bosentan or a pharmaceutically acceptable salt thereof, in which bosentan has a purity of about 99.8% to about 99.99% and comprising (p tert-butyl-N-[6-hydroxy-5-(2-methoxyphenoxy)-2-(2-pyrimidinyl)-4-pyrimidinyl]benzenesulfonamide) (deshydroxyethyl bosentan impurity) in an amount of less than about 0.15% as measured by HPLC.

46-47. (canceled)

48. Bosentan of claim 45, comprising deshydroxyethyl bosentan impurity in an amount of about 0.01% to about 0.15%.

49. (canceled)

50. Bosentan of claim 45, essentially free of deshydroxyethyl bosentan impurity.

51. Bosentan of claim 45, further comprising 1,2-bis[[5-(2-methoxyphenoxy)-2-pyrimidin-2yl-pyrimidin-4yl]-4-tert-butyl-benzenesulfonamide]ethanediol (bosentan dimer impurity) in an amount of less than about 0.1% as measured by HPLC.

52. Bosentan of claim 51, comprising bosentan dimer impurity in an amount of less than about 0.05%.

53. Bosentan of claim 45, essentially free of bosentan dimer impurity.

54. A purification process for obtaining bosentan or a pharmaceutically acceptable salt thereof of claim 45, comprising:
a) forming a solution of crude bosentan in a solvent medium comprising ethyl acetate and methanol;
b) optionally, filtering the solvent solution to remove any extraneous matter; and
c) isolating highly pure bosentan substantially free of the deshydroxyethyl bosentan impurity from the solution, and optionally converting the highly pure bosentan obtained into its pharmaceutically acceptable salts thereof.

55-57. (canceled)

58. The process of claim 54, wherein the methanol is used in an amount of about 0.5 to 6.0 volumes with respect to ethyl acetate.

59. The process of claim 58, wherein the methanol is used in an amount of about 2.0 to 3.0 volumes with respect to ethyl acetate.

60-89. (canceled)

90. The pharmaceutical composition of claim 1, wherein the bosentan crystalline Form A₅ has a D₉₀ particle size of less than or equal to about 200 microns; less than or equal to about 100 microns; or less than or equal to about 15 microns.

91-97. (canceled)
